(19) United States
(12) Patent Application Publication
(10) Pub. No.: US 2024/0007294 A1
(43) Pub. Date: Jan. 4, 2024

(54) EMBEDDED SERVICING AND AUTHENTICATION FOR MEDICAL DEVICE

(71) Applicant: Welch Allyn, Inc., Skaneateles Falls, NY (US)

(72) Inventors: Thomas Henry Briggs, Shippensburg, PA (US); Brian Moons, Beaverton, OR (US); John R. Vann, St. Thomas (CA)

(21) Appl. No.: 18/341,005

(22) Filed: Jun. 26, 2023

Related U.S. Application Data
(60) Provisional application No. 63/367,501, filed on Jul. 1, 2022.

(51) Int. Cl. H04L 9/32; H04L 9/40

(52) U.S. Cl. CPC H04L 9/3234; H04L 9/3263; H04L 63/101

(57) ABSTRACT

A medical device for assessing a health status of a patient is described. The medical device includes firmware including service software for servicing the one or more sensor modules, the service software including an embedded web server, and clinical software for acquiring the one or more physiological parameter measurements from the patient. The medical device establishes a server-client communications connection with a web browser on a client device. The medical device provides access on the web browser to the service software through the server-client communications connection. The access to the service software is provided without loading the service software onto the client device.

### BACKGROUND

Caregivers typically use various types of healthcare equipment to provide healthcare to a patient in a clinical care environment. The healthcare equipment can include multifunction medical devices that perform more than one function during patient assessment. Such multifunction medical devices are typically used to measure one or more physiological parameters from a single patient or from multiple patients within the clinical care environment. Also, such multifunction medical devices typically include various types of sensor modules and peripheral components for measuring the one or more physiological parameters.

These types of medical devices need to be serviced at periodic intervals. For example, sensors, probes, and other peripheral components connected to the multifunction medical devices can have a defined useful life before these components need to be replaced. Likewise, the software and firmware running on the medical devices may need to be periodically updated.

### SUMMARY

In general terms, the present disclosure relates to a medical device that includes embedded servicing. In one possible configuration, the medical device exhibits a technical effect by providing access on a web browser of a client device to service software without loading the service software onto the client device. In another possible configuration, the medical device exhibits a technical effect by determining an accessibility level for the service software based on a signed certificate for a user in possession of an authentication device. Various aspects are described in this disclosure, which include, but are not limited to, the following aspects.

One aspect relates to a medical device for assessing a health status of a patient, the medical device comprising: one or more sensor modules each configured to obtain one or more physiological parameter measurements from the patient; at least one processing device; and a memory device storing: firmware including: service software for servicing the one or more sensor modules, the service software including an embedded web server; and clinical software for acquiring the one or more physiological parameter measurements from the patient; and instructions which, when executed by the at least one processing device, cause the at least one processing device to: establish a server-client communications connection with a web browser on a client device; and provide access on the web browser to the service software through the server-client communications connection, the access to the service software being provided without loading the service software onto the client device.

Another aspect relates to a medical device for assessing a health status of a patient, the medical device comprising: at least one processing device; and a memory device storing instructions which, when executed by the at least one processing device, cause the at least one processing device to: receive a password assigned to an authentication device; receive a signed certificate stored on the authentication device, the signed certificate created by a certificate authority; validate the signed certificate by using a public key stored on the memory device; determine an accessibility level based on the signed certificate; establish a server-client communications connection between an embedded web server installed on the medical device and a web browser installed on a client device; and provide access on the web browser to service software installed on the medical device through the server-client communications connection, the accessibility level determines a level of access on the web browser to the service software.

Another aspect relates to a method of authenticating a user for servicing a medical device, the method comprising: activating an authentication device; receiving a signed certificate stored on the authentication device; validating the signed certificate by using a public key; and when the signed certificate is validated, determining an accessibility level based on the signed certificate for the user; establishing a server-client communications connection between an embedded web server installed on the medical device and a web browser installed on a client device; and providing access on the web browser to service software installed on the medical device through the server-client communications connection, wherein the accessibility level determines a level of access on the web browser to the service software.

### DESCRIPTION OF THE FIGURES

The following drawing figures, which form a part of this application, are illustrative of the described technology and are not meant to limit the scope of the disclosure in any manner.

FIG. 1 schematically illustrates an example of a system for collecting physiological parameter measurements from patients in a clinical care environment.

FIG. 2 illustrates an example of a medical device of the system of FIG. 1.

FIG. 3 schematically illustrates an example of a kit for servicing the medical device of FIG. 2.

FIG. 4 schematically illustrates an example of device firmware installed on the medical device of FIG. 2.

FIG. 5 schematically illustrates an example of a method of servicing the medical device of FIG. 2.

FIG. 6 schematically illustrates an example of a method of configuring an authentication device for servicing the medical device FIG. 2.

FIG. 7 schematically illustrates an example of an authentication device following completion of the method of FIG. 6.

FIG. 8 schematically illustrates an example of a public key infrastructure hierarchy for providing access to service software installed on the medical device of FIG. 2.

FIG. 9 schematically illustrates an example of a method of authenticating a user for servicing the medical device of FIG. 2.

FIG. 10 schematically illustrates an example of the medical device of FIG. 2.

### DETAILED DESCRIPTION

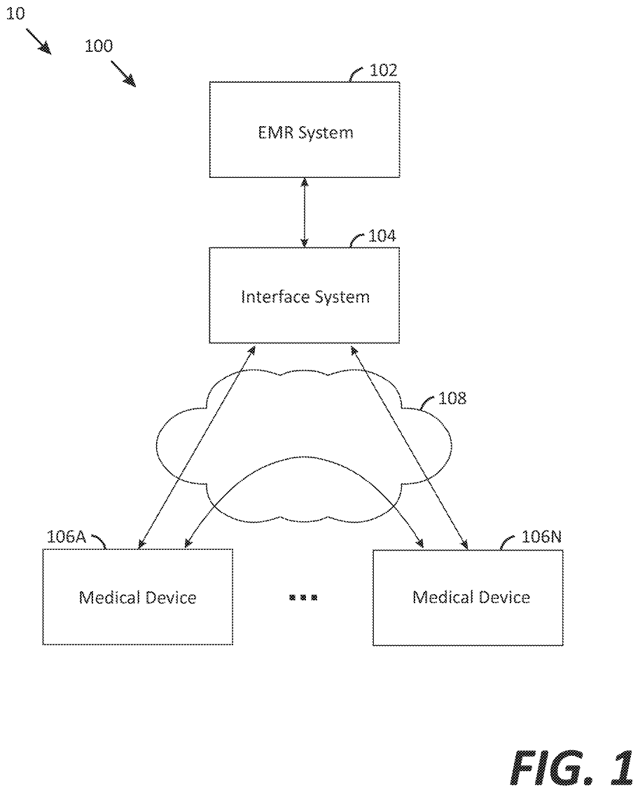

FIG. 1 schematically illustrates an example of a system 100 for collecting physiological parameter measurements from patients in a clinical care environment 10. An example of the clinical care environment can include a healthcare facility such as a hospital, a surgical center, a nursing home, a long term care facility, or similar type of facility. As shown in FIG. 1, the system 100 includes an Electronic Medical Records (EMR) system 102, an interface system 104, medical devices 106A-106N, and a network 108.

The network 108 is a communications network that facilitates data communication between the medical devices 106A-106N, and between the medical devices 106A-106N and the interface system 104. The network 108 can include a set of computing devices and links between the computing devices. The computing devices in the network 108 use the links to enable data communication among the computing devices in the network. The network 108 can include routers, switches, mobile access points, bridges, hubs, intrusion detection devices, storage devices, standalone server devices, blade server devices, sensors, desktop computers, firewall devices, laptop computers, tablet computers, handheld computers, smartphones, and other types of computing devices. In various embodiments, the network 108 includes various types of links. For example, the network 108 can include wired and/or wireless links. In some embodiments, the network 108 is implemented at various scales. For example, the network 108 can be implemented as one or more local area networks (LANs), metropolitan area networks, subnets, wide area networks (such as the Internet), or can be implemented at another scale.

The EMR system 102 is a computing system that allows storage, retrieval, and manipulation of electronic medical records. As used herein, a computing system is a system of one or more computing devices. A computing device is a physical, tangible device that processes data. Example types of computing devices include personal computers, standalone server computers, blade server computers, mainframe computers, laptop computers, tablet computers, handheld computers, smartphones, and other types of electronic devices that process data.

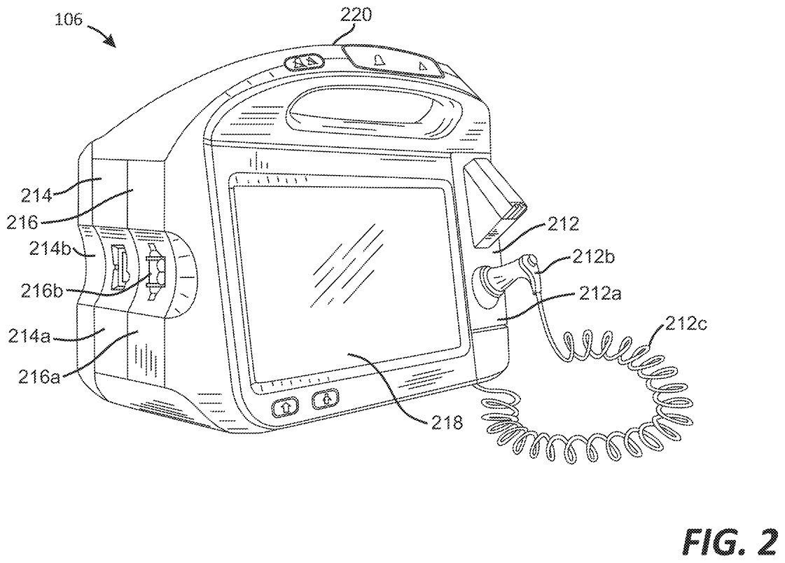

Each of the medical devices 106 is a computing device. The medical devices 106 can provide various types of functionalities. For example, the medical devices 106 can include a physiological parameter monitoring platform or spot monitor, such as the one illustrated in FIG. 2. In such examples, the physiological parameter monitoring platform or spot monitor can be used by a clinician to measure and/or monitor physiological parameters of multiple patients, and to display representations of the measured physiological parameters.

In further examples, the medical devices 106 can include any type of physical assessment device such as, without limitation, ophthalmoscopes, otoscopes, dermatoscopes, vision screeners, and the like. The medical devices 106 can further include other types of devices that are capable of measuring physiological parameters such as hospital beds. The medical devices 106 can communicate with each other through the network 108.

The interface system 104 is a computing system that acts as an interface between the EMR system 102 and the medical devices 106. In some embodiments, the interface system 104 includes Connex® Vitals Management Software from Hillrom of Batesville, Indiana.

As an illustrative example, the interface system 104 provides a single software interface for each of the medical devices 106 such that the medical devices can send requests to the software interface provided by the interface system 104. When the interface system 104 receives a request from one of the medical devices 106, the interface system 104 translates the request into a request that works with the software interface provided by the EMR system 102. The interface system 104 then provides the translated request to the EMR system 102. When the interface system 104 receives a response from the EMR system 102, the interface system 104 translates the response into a format understood by the medical devices 106, and then forwards the translated response to an appropriate one of the medical devices 106. In this manner, the interface system 104 allows use of the medical devices 106 in various types of EMR systems.

The medical devices 106 can send various types of data to the interface system 104 for storage in the EMR system 102 and can receive various types of data from the EMR system 102 through the interface system 104. For example, a medical device 106 can send physiological parameter measurements and clinical observations to the interface system 104 for storage in the EMR system 102. In further examples, a medical device 106 can retrieve past physiological parameter measurements, clinical observations, lab results, scans, and other patient health information from the EMR system 102 through the interface system 104.

FIG. 2 illustrates an example of a medical device 106 of the system 100. In this example, the medical device 106 is a physiological parameter monitoring platform or spot monitor. The medical device 106 can include one or more sensor modules. Each of the sensor modules is configured to measure one or more physiological parameters of a patient.

In the illustrative example shown in FIG. 2, the medical device 106 includes a temperature sensor module 212 that is accessible from a front side of the device, and a photoplethysmogram sensor module 214 and a non-invasive blood pressure (NIBP) sensor measurement module 216 that are accessible from a left hand side of the device. As used herein, a “module” is a combination of physical structure which resides in the medical device 106 and peripheral components that attach to and reside outside of the medical device 106. The medical device 106 can include additional sensor modules for receiving additional physiological parameter measurements, including ECG or EKG measurements.

The temperature sensor module 212 is designed to receive body temperature measurements of a patient. As an illustrative example, the temperature sensor module 212 includes a front panel 212a that has an outer surface accessible from the front side of the medical device 106. The front panel 212a provides access to a wall (not shown) storing a removable probe (not shown), also referred to as a temperature probe, attached to a probe handle 212b. The temperature probe and the probe handle 212b are tethered to the temperature sensor module 212 via an insulated conductor 212c. The temperature probe is configured to make physical contact with the patient in order to sense the body temperature of the patient.

The photoplethysmogram sensor module 214 can be used to measure blood oxygen saturation and pulse. Also, the photoplethysmogram sensor module 214 can be used to measure respiration rate based on changes in a plethysmography waveform. The photoplethysmogram sensor module 214 includes a front panel 214a having a connector port 214b that enables a connection between a pulse oximeter (not shown) and the photoplethysmogram sensor module 214 residing inside the medical device 106. The pulse oximeter resides externally, and is configured to interoperate with the photoplethysmogram sensor module 214 when connected to the photoplethysmogram sensor module 214 via the connector port 214b. The pulse oximeter can include a clip that attaches to an appendage (e.g., finger) of the patient. The clip includes an infrared light transmitter, and a sensor that detects and measures blood oxygen saturation and pulse rate based on transmission of the infrared light through the patient's appendage.

The NIBP sensor module 216 can be used to measure blood pressure of the patient. The NIBP sensor module 216 includes a front panel 216a having a connector port 216b that enables a connection between one or more peripheral NIBP components and the NIBP sensor module 216 residing inside the medical device 106. The peripheral NIBP components reside externally, and are configured to interoperate with the NIBP sensor module 216 when connected to the NIBP sensor module 216 via the connector port 216b. The peripheral NIBP components can include an inflatable cuff that attaches to an appendage of the patient, such as an upper arm. The inflatable cuff is used to measure systolic and diastolic blood pressure of the patient, mean arterial pressure (MAP) of the patient, and pulse rate of blood flowing within the patient.

As shown in FIG. 2, a front side of the medical device 106 includes a display screen 218. In this example, the display screen 218 is a built-in display. The display screen 218 can display graphical representations of the physiological parameter measurements received from the various sensor modules of the medical device 106 including the temperature sensor module 212, the photoplethysmogram sensor module 214, and the NIBP sensor module 216. The display screen 218 can further display graphical representations of additional physiological parameter measurements and clinical observations from additional sources such as from other medical devices and the EMR system 102 via connections through the network 108. In some examples, the display screen 218 is a touchscreen that receives manual inputs from a user of the medical device 106. Also, the medical device 106 can include one or more handles 220 on its housing that enable the medical device 106 to be carried by hand by the user.

The medical device 106 is able to operate within one or more workflows. A workflow is a series of one or more tasks that a user of the medical device 106 performs. When the medical device 106 operates within a workflow, the medical device 106 provides functionality suitable for assisting the user (e.g., caregiver) in performing the workflow. When the medical device 106 operates within different workflows, the medical device 106 provides different functionality.

When the medical device 106 is manufactured, the medical device 106 is configured to be able to operate within one or more predefined workflows. After the medical device 106 is manufactured, the medical device 106 can be reconfigured to operate within one or more additional and/or customized workflows. In this way, the clinical care environment 10 (such as a hospital) can adapt the medical device 106 for use in customized workflows as desired.

As an illustrative example, the medical device 106 can operate in a monitoring workflow or in a non-monitoring workflow. A monitoring workflow can include continuously monitoring the physiological parameters of a single patient. Example types of non-monitoring workflows include a spot check workflow and/or a triage workflow.

When the medical device 106 operates in the monitoring workflow, the medical device 106 obtains a series of measurements of one or more physiological parameters (e.g., temperature, SpO2, blood pressure, EKG, and the like) of a single patient over a period of time. In addition, the medical device 106 displays, on the display screen 218, a monitoring workflow home screen. The monitoring workflow home screen can include a representation of the physiological parameters measured from the patient who is being monitored.

As an illustrative example, when the medical device 106 operates in the monitoring workflow, the medical device 106 can obtain a blood pressure measurement of a single patient once every ten minutes for six hours. The medical device 106 can display a monitoring workflow home screen on the display screen 218 that displays a representation of the blood pressure measurements measured from the patient. In this way, the medical device 106 allows a user such as caregiver in the healthcare facility to monitor a health status of the patient.

When the medical device 106 operates within a non-monitoring workflow, the medical device 106 obtains measurements of one or more physiological parameters from a series of patients that have been previously identified. As used herein, a patient is previously identified when the medical device 106 stores information regarding the identity of the patient. The medical device 106 can display a non-monitoring workflow home screen on the display screen 218 that displays a representation of a physiological parameter measured from a previously identified patient in the series of patients. In this way, the medical device 106 allows a user such as caregiver in the healthcare facility to check a health status of multiple patients.

In another example, when the medical device 106 operates in a triage workflow, the medical device 106 can obtain measurements of physiological parameters from each patient in a series of unidentified patients, such as when the patients arrive in an emergency department of a hospital. In this example, the medical device 106 displays a triage workflow home screen that displays a representation of a physiological parameter measured from a patient who has not been previously identified. In this way, the medical device 106 can perform triage on a series of unidentified patients as they arrive. As used herein, a patient is an unidentified patient when the medical device 106 does not store information regarding the identity of the patient.

The monitoring workflow home screen can be different from the non-monitoring workflow home screen in various ways. For example, the monitoring workflow home screen can include at least one user-selectable control that is not included in the non-monitoring workflow home screen and/or the non-monitoring workflow home screen can include at least one user-selectable control that is not included in the monitoring workflow home screen. As another example, different navigation options can be provided in the different workflows to allow for more efficient navigation within the respective workflows based on their particular functions and/or needs. Additional examples of the differences between the monitoring workflow home screen and the non-monitoring workflow home screen are contemplated.

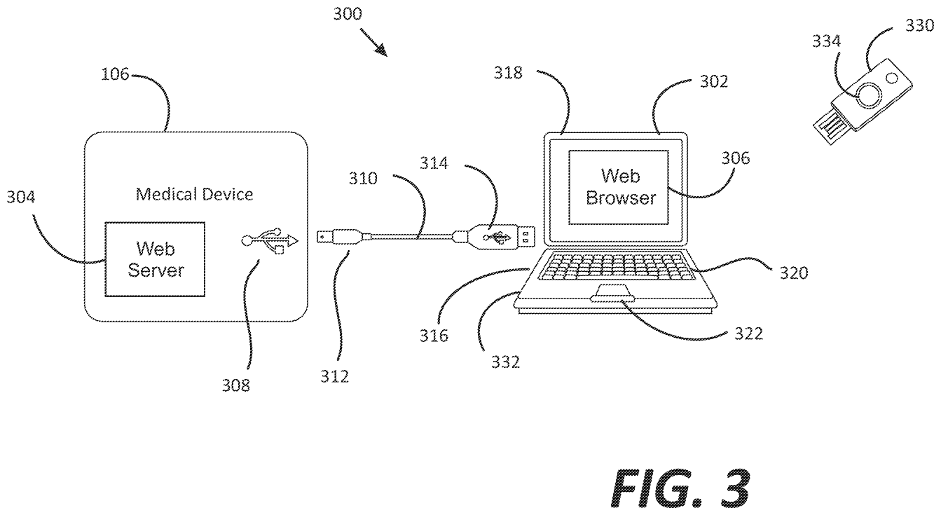

FIG. 3 schematically illustrates an example of a kit 300 for servicing the medical device 106. Illustrative examples of the servicing that can be provided on the medical device 106 can include, without limitation, calibration (including generating signed calibration certificates), functional verification tests, accuracy checks, component reporting, log file display and download, debugging, factory reset and/or decommission, and the like.

The kit 300 includes a client device 302, which can include any type of internet enabled device that allows for secure internet communications protocols such as hypertext transfer protocol secure (HTTPS). Illustrative examples of the client device 302 can include, without limitation, a laptop, a tablet computer, a smartphone, and other computing device.

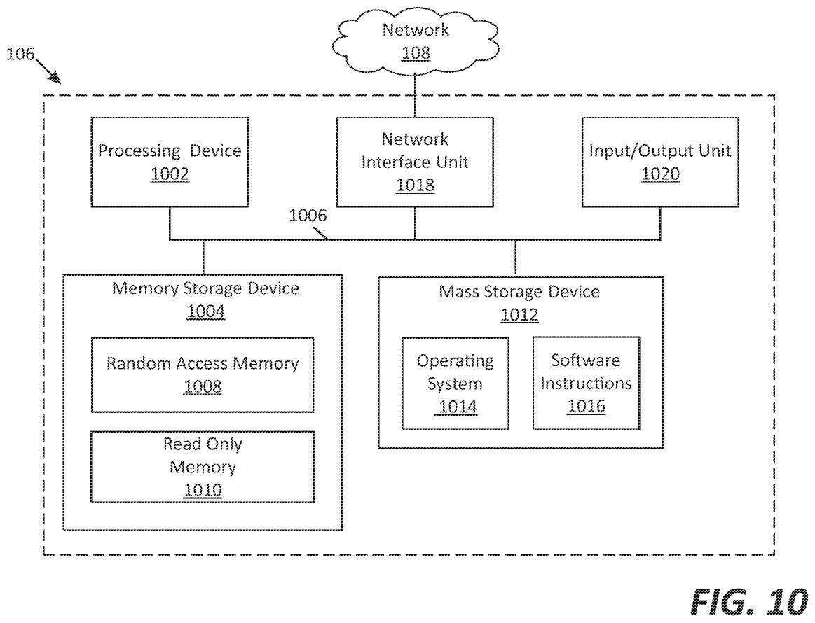

The medical device 106 includes an embedded web server 304. In some example embodiments, the embedded web server 304 is stored on a memory device of the medical device 106. In some examples, the medical device 106 is manufactured as a unified deliverable having the embedded web server 304 installed thereon before delivery to a customer such as an operator of the clinical care environment 10. Examples of various types of memory devices on the medical device 106 will be described in more detail below with respect to FIG. 10.

In the illustrative example shown in FIG. 3, the kit 300 further includes a cable 310 having first and second connectors 312, 314. In this illustrative example, the medical device 106 includes a first port 308 that receives the first connector 312 of the cable 310, and the client device 302 includes a second port 316 that receives the second connector 314 of the cable 310 for providing a physical connection for a server-client communications connection between the embedded web server 304 installed on the medical device 106 and a web browser 306 that can be viewed on a display screen 318 of the client device 302. In this illustrative example, the cable 310 is a universal serial bus (USB) cable, and the first and second ports 308, 316 are USB ports. The first and second ports 308, 316 can include, without limitation, USB-A, USB-C, Lightning, and other types of connector ports. The cable 310 can provide an Ethernet or similar type of connection between the medical device 106 and the client device 302 for carrying Internet Protocol (IP) communications between the medical device 106 and the client device 302.

In some examples, the medical device 106 and the client device 302 wirelessly connect with one another for providing a server-client communications connection between the embedded web server 304 installed on the medical device 106 and the web browser 306 that can be viewed on the display screen 318 of the client device 302. The wireless connection can be used to carry IP communications between the medical device 106 and the client device 302. Illustrative examples of wireless connections between the medical device 106 and the client device 302 can include, without limitation, Wi-Fi, Bluetooth, wireless local area network (WLAN) links, ZigBee, ultra-wideband (UWB), and other wireless technologies.

The embedded web server 304 installed on the medical device 106 establishes the server-client communications connection with the web browser 306 installed on the client device 302 to facilitate servicing the medical device 106. For example, the medical device 106 is able to send live status and telemetry data to the web browser 306, which can display a user interface on the display screen 318 for viewing by a user to drive calibration workflows. The medical device 106 can conduct and report all servicing actions through code behind the embedded web server 304 without requiring specialized software to be loaded onto the client device 302. This eliminates the need to maintain and update specialized software on the client device 302.

In the example shown in FIG. 3, the client device 302 is a laptop computer that includes input devices, such as a keyboard 320 and a touch pad 322, that allow a user of the client device 302 (e.g., biomedical equipment technician) to operate the service software applications installed on the medical device 106 via the server-client communications connection. In some instances, the input devices of the client device 302 allows the user of the client device 302 (e.g., biomedical equipment technician) to control the one or more sensor modules on the medical device 106 via the server-client communications connection.

The server-client communications connection allows the client device 302 to service the medical device 106 without requiring the client device 302 to have separate service software installed thereon. This eliminates the need for having service software installed on the client device 302 for servicing the medical device 106, which must be created and maintained separately from the service software applications installed on the medical device 106. This can be especially advantageous given that maintaining separate service software applications on the client device 302 can require careful managing of device compatible firmware versions.

The embedded web server 304 does not cause any service software to be loaded onto the client device 302 for servicing the medical device 106. Instead, the web browser 306 can be any type of web browser that supports programming language such as JavaScript that allows for connection with the embedded web server 304 on the medical device 106. Thus, the web browser 306 allows a user of the client device 302 to access service software applications installed on the medical device 106 without requiring any specialized service software applications to be installed on the client device 302. Illustrative examples of the web browser 306 can include, without limitation, Google Chrome, Microsoft Edge, Mozilla Firefox, Safari, Internet Explorer, and other types of browsers that can support the server-client communications connection.

In some examples, the server-client communications connection includes a physical connection between the medical device 106 and the client device 302 such as through the cable 310. In some further examples, the server-client communications connection includes a wireless connection between the medical device 106 and the client device 302 such as through Wi-Fi, Bluetooth, WLAN, ZigBee, UWB, and other wireless communications protocols.

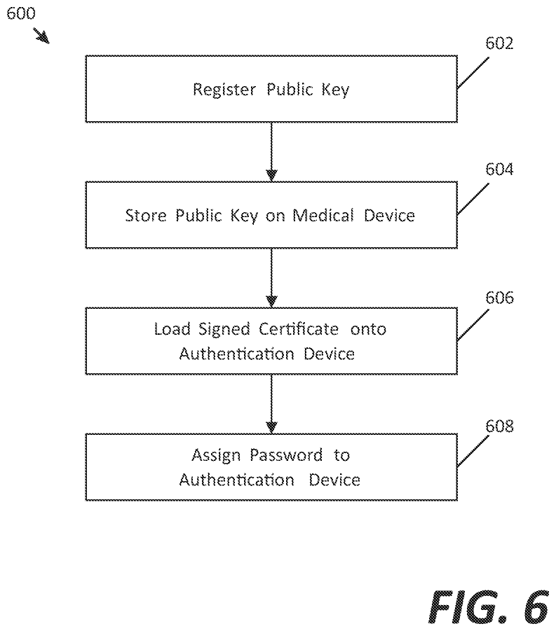

As further shown in FIG. 3, the kit 300 can further include an authentication device 330 for authenticating a user of the client device 302 such as a biomedical equipment technician who is authorized to service the medical device 106. In some examples, the authentication device 330 can be plugged into a third port 332 on the client device 302 for authenticating the user of the client device. In some examples, the third port 332 is a USB-A, USB-C, Lightning, or other type of connector port. In alternative examples, the authentication device 330 can be plugged into the second port 316 on the client device 302 for authenticating the user of the client device. In further alternative examples, the authentication device 330 can be plugged into the first port 308 of the medical device 106 for authenticating the user of the client device. In further examples, the authentication device 330 can connect to the client device 302 or to the medical device 106 through a wireless interface such as near-field communication (NFC) or other similar type of proximity technology that is recognizable by the medical device 106 and/or client device 302. The authentication device 330 will be described in more detail with reference to FIGS. 6-9.

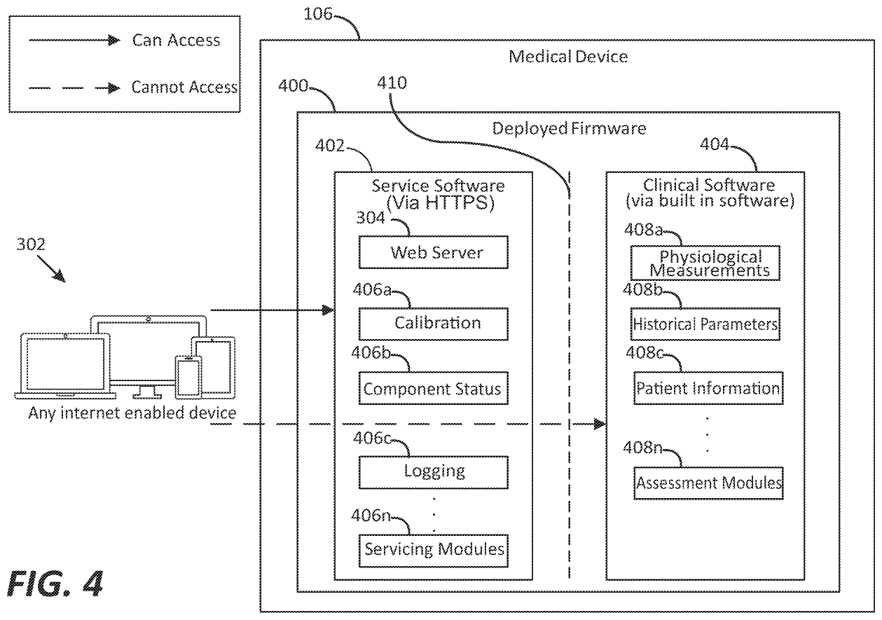

FIG. 4 schematically illustrates an example of device firmware 400 installed on the medical device 106. The device firmware 400 includes service software 402 for servicing the one or more sensor modules on the medical device 106, and clinical software 404 for assessing a health status of one or more patient being monitored by the medical device 106.

As shown in FIG. 4, the service software 402 is isolated from the clinical software 404. This is schematically represented by a barrier 410, which is shown separating the service software 402 from the clinical software 404. In some examples, the barrier 410 is a firewall.

One or more client devices 302 (i.e., any type of internet enabled device including laptops, tablet computers, smartphones, and other computing devices) are able to access the service software 402. In accordance with the examples described above, the client devices 302 can access the service software 402 via the server-client communications connection established by the embedded web server 304 installed on the medical device 106 (see FIG. 3).

As further shown in FIG. 4, the client devices 302 are prevented and/or blocked from accessing the clinical software 404. By preventing access to the clinical software 404, the one or more client devices 302 are prevented from having access to medical data on the medical device 106. For example, the one or more client devices 302 cannot receive medical data such as protected health information, which includes information about a health status (e.g., physiological parameter measurements, clinical observations, lab results, etc.), provision of healthcare, or payment for healthcare collected by the clinical care environment 10, and that can be linked to specific individuals, such as patients monitored by the medical device 106.

The service software 402 can include the embedded web server 304, and one or more service software applications 406 for servicing the medical device 106. As an illustrative example, the service software 402 can include calibration applications 406a for calibrating the one or more sensor modules of the medical device 106, component status applications 406b for checking the status of one or more peripheral components of the medical device 106, logging applications 406c for displaying and downloading log files, and additional types of service software applications 406n for performing various servicing functions on the medical device 106. The service software applications 406 do not obtain or include protected health information.

As further shown in FIG. 4, the clinical software 404 can include one or more applications 408 for performing various functions related to assessing a health status of a patient. For example, the clinical software 404 can include physiological parameter applications 408a for receiving and displaying graphical representations of physiological parameter measurements received from peripheral components and sensors connected to the medical device 106, historical physiological parameter applications 408b for receiving and displaying historical physiological parameter measurements such as from the EMR system 102, patient information applications 408c for receiving and/or displaying patient information such as patient name, date of birth, patient medical record number, etc., and additional types of patient assessment applications 408n. In this example, one or more of the applications 408 within the clinical software 404 can include protected health information belonging to one or more patients.

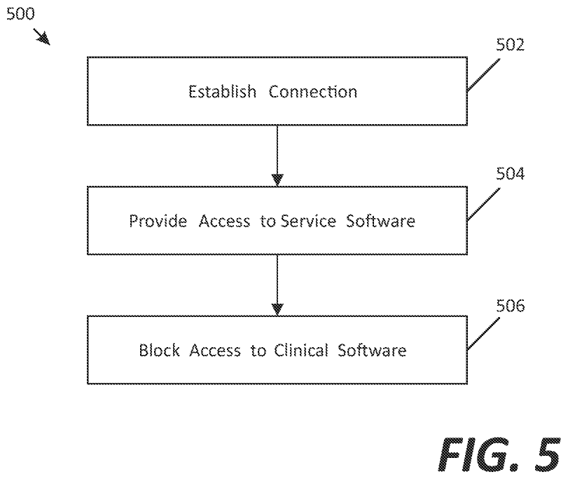

FIG. 5 schematically illustrates an example of a method 500 of performing servicing on the medical device 106. In some example embodiments, the method 500 can be performed by the embedded web server 304 installed on the medical device 106.

The method 500 includes an operation 502 of establishing the server-client communications connection with the web browser 306 installed on a client device 302. In some examples, the server-client communications connection is established through a physical connection such as by plugging the first connector 312 of the cable 310 into the first port of 308 of the medical device 106, and by plugging the second connector 314 of the cable 310 into the second port 316 of the client device 302. In such examples, the embedded web server 304 can identify and/or recognize the client device 302 once the physical connection is made between the medical device 106 and the client device 302, and the embedded web server 304 then establishes the server-client communications connection with the web browser 306 on the client device 302.

In other examples, the server-client communications connection is established through a wireless connection such as through Wi-Fi, Bluetooth, WLAN, ZigBee, UWB, and other wireless communications protocols. In such examples, the embedded web server 304 can identify and/or recognize the client device 302 wirelessly, and the embedded web server 304 can thereafter send a request to the client device 302 to establish the server-client communications connection with the web browser 306 on the client device 302. Operation 502 does not include loading any specialized software onto the client device 302 for servicing the medical device 106.

The method 500 includes an operation 504 of providing access on the web browser 306 to one or more of the service software applications 406 installed on the medical device 106. The one or more service software applications 406 can be used to perform various servicing functions on the medical device 106 such as, without limitation, calibrating the sensor modules, functional verification tests, accuracy checks, component reporting, and log file display and download, debugging, factory reset and/or decommission, and the like. In operation 504, the embedded web server 304 can provide bi-directional communication between the client device 302 and the service software 402 installed on the medical device 106 without requiring any specialized service software to be loaded onto the client device 302.

The method 500 further includes an operation 506 blocking or preventing the web browser 306 from accessing the clinical software 404 installed on the medical device 106. As described above, the clinical software 404 includes one or more applications 408 that can include protected health information. Operation 506 prevents the client device 302 from acquiring the protected health information, whether done intentionally or unintentionally. In some examples, operation 506 is performed by isolating the service software 402 from the clinical software 404, such that the embedded web server 304 is not able to provide the web browser 306 with access to the one or more applications 408 of the clinical software 404.

As shown in FIG. 4, the one or more client devices 302 are able to access the service software 402 via the server-client communications connection established by the embedded web server 304 installed on the medical device 106 for viewing one or more of the service software applications 406 on the web browser 306. As will now be described in more detail, the medical device 106 includes features to restrict the access to the service software 402 stored on the medical device 106 to protect the security of the medical device 106 from malicious users, and also the confidentiality of protected health information on the medical device 106.

Additionally, in some instances, the medical device 106 may not be connected to a local area network (LAN) such as the network 108 (see FIG. 1). In such instances, the medical device 106 cannot use standard authentication servers to authenticate users who request access to the service software 402 on the medical device. As will be explained in more detail, the features included on the medical device 106 enable the medical device 106 to identify, authenticate, and authorize users of the client device 302 independently of whether or not the medical device 106 is connected to a network to ensure that only authorized personnel are given access to the service software applications 406 for servicing the medical device 106.

As used herein, identification includes identifying a user who requests access to one or more of the service software applications 406 installed on the medical device 106. In some examples, the medical device 106 identifies a user who requests access to the service software 402, and monitors the actions of the user once they are identified.

As used herein, authentication includes establishing that the identified user is who they claim to be. Best practices indicate that storing simple passwords is insufficient for authentication of users given present security threats such as phishing attacks.

As used herein, authorization includes determining what functions and services an authenticated user is permitted to perform which can be restricted based on geographical location, level of training, and other qualifications. Authorization can include associating a specific user, based on their credentials, with a set of permitted actions. For example, a manufacturer of the medical device 106 may authorize representatives to service all of the medical devices 106, or only a certain fleet of the medical devices 106 based on a particular geographical region. As another illustrative example, a healthcare delivery organization (HDO) may authorize a biomedical equipment technician to service the medical devices 106 that are used in a specific healthcare facility, or that are used in a particular department or floor within the facility. As a further illustrative example, a manufacturer of the medical devices 106 may only authorize a biomedical equipment technician to service the medical devices 106 after the technician completes appropriate training or otherwise possesses sufficient credentials.

As described above, the kit 300 (see FIG. 3) can include an authentication device 330 for identifying, authenticating, and authorizing a user of the client device 302 for servicing the medical device 106. FIG. 6 schematically illustrates an example of a method 600 of configuring the authentication device 330 for servicing the medical device 106. In some examples, the method 600 is performed by a manufacturer of the medical device 106 to ensure that only authorized personnel are permitted to access the service software applications 406. In some examples, the method 600 follows a public key infrastructure (PKI) process.

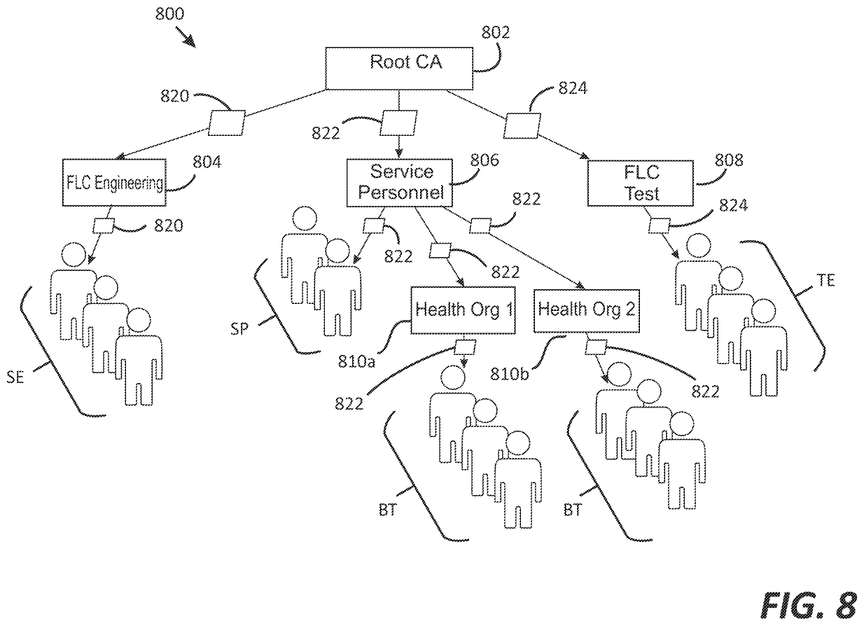

The method 600 includes an operation 602 of registering a public key with a certificate authority. The certificate authority stores, signs, and issues signed certificates. In some examples, the certificate authority issues signed certificates based on a hierarchy, such as the hierarchy 800 shown in FIG. 8, which will be described in more detail below. The public key validates the signed certificates issued by the certificate authority to authenticate users. In some examples, the manufacturer of the medical device 106 manages the certificate authority.

Next, the method 600 includes an operation 604 of storing the public key onto the medical device 106. The public key can be stored a memory storage device 1004 and/or on a mass storage device 1012 installed on the medical device 106, as shown in FIG. 10.

Next, the method 600 includes an operation 606 of loading a signed certificate onto the authentication device 330. As shown in FIG. 3, the authentication device 330 resembles a flash drive (also called a thumb drive), which is a data storage device that includes a memory, such as flash memory, with an integrated connection interface. The memory of the authentication device 330 is rewritable such that the authentication device 330 can be updated to store new signed certificates. The authentication device 330 is portable, and can be removably plugged into and out of one or more ports on the medical device 106 and/or on the client device 302.

Next, the method 600 includes an operation 608 of assigning a password to the authentication device 330. The password is configured to unlock the authentication device 330 for recognition by the medical device 106. In some examples, an initial password is created by the manufacturer of the medical device 106 and is assigned to the authentication device 330, and after the authentication device 330 is delivered, the password can be changed by a user.

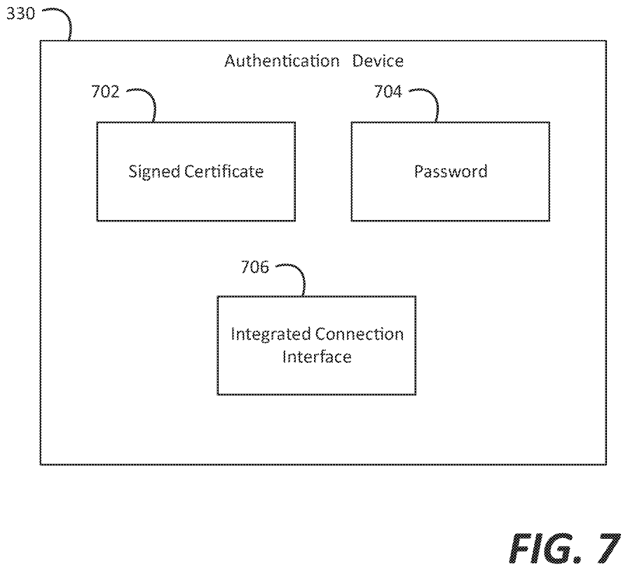

FIG. 7 schematically illustrates an example of the authentication device 330 after completion of the method 600. The authentication device 330 stores a signed certificate 702 and a password 704. The authentication device 330 further includes an integrated connection interface 706. In some examples, the integrated connection interface 706 includes a plug interface such as a USB-A, USB-C, Lightning, or other type of physical interface. In some examples, the integrated connection interface 706 includes a wireless interface such as near-field communication (NFC) that is recognizable by the medical device 106 and/or client device 302.

FIG. 8 schematically illustrates an example of a hierarchy 800 of a public key infrastructure (PKI) for providing access to the service software applications 406 installed on the medical device 106. The hierarchy 800 is provided as an illustrative example and for illustration purposes only. As shown in FIG. 8, a certificate authority (CA) 802 can issue signed certificates for various entities and individuals to service the medical device 106. As described above, the CA 802 can be managed by the manufacturer of the medical device 106.

In this illustrative example, the CA 802 issues a first type of signed certificate 820 to a service engineering department 804, a second type of signed certificate 822 to a service personnel department 806, and a third type of signed certificate 824 to a software testing and debugging department 808. Each of the service engineering department 804, the service personnel department 806, and the software testing and debugging department 808 can be departments within the manufacturer of the medical device 106. Each of the service engineering department 804, the service personnel department 806, and the software testing and debugging department 808 can distribute their respective signed certificates to their individual members such as service engineers SE, service personnel SP, and testing engineers TE.

In this illustrative example, the service personnel department 806 issues the second type of signed certificate 822 to one or more customers such as first and second healthcare organizations 810a, 810b. As further shown, the first and second healthcare organizations 810a, 810b can each separately issue the second type of signed certificate 822 to one or more of their respective employees such as biomed equipment technicians BT.

Each of the different types of signed certificates can provide a different level of access to the embedded web server 304, the service software 402, and the service software applications 406 based on the credentials of a user. In further examples, the signed certificates can define a date range during which the signed certificates are valid, and after which, the signed certificates are expired. This can be done to verify that the signed certificates remain effective and/or authorized by the CA 802. The signed certificates when stored on the authentication device 330 allow the medical device to identify, authenticate, and authorize the various users without requiring a network connection such as to the internet.

As an illustrative example, the first and second types of signed certificates 820, 822 provide the service engineers SE and the biomed equipment technicians BT access to a standard set of the service software applications 406 for servicing the medical device 106, while the third type of signed certificate 824 provides a higher level of access for the testing engineers TE. For example, the higher level of access can allow the testing engineers TE to perform testing and debugging of the device firmware 400, which is not allowed to be performed by the service engineers SE and biomed equipment technicians BT. Additional examples how the signed certificates can provide different levels of access to the device firmware 400 are possible.

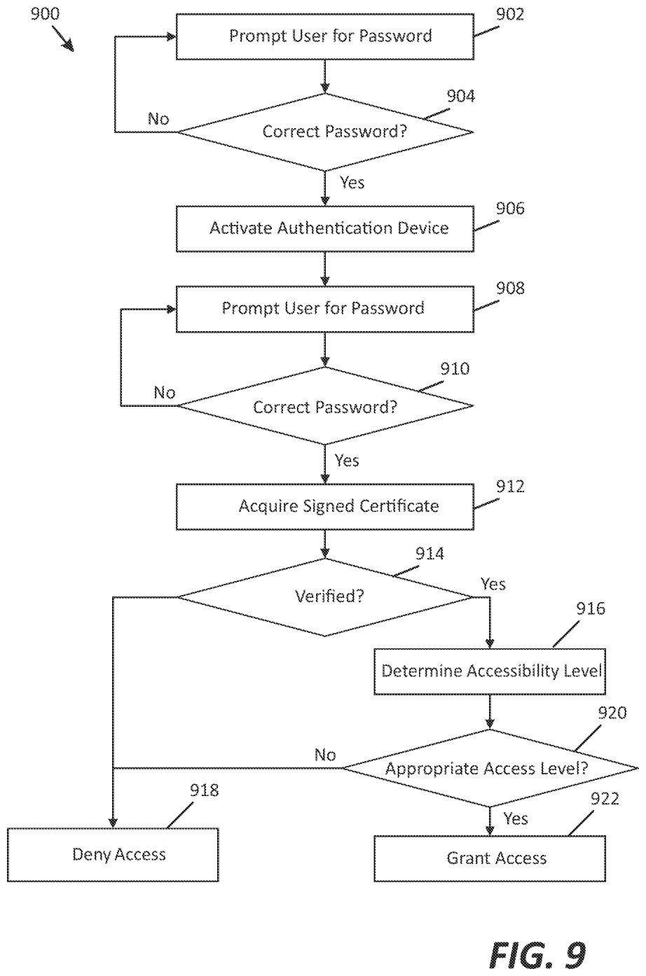

FIG. 9 schematically illustrates an example of a method 900 of authenticating a user for servicing the medical device 106. In some examples, the method 900 is performed by the medical device 106. In some examples, the method 900 is performed before the server-client communications connection is established between the embedded web server 304 installed on the medical device 106 and the web browser 306 on the client device 302. Accordingly, the method 900 can be performed by the medical device 106 to authenticate a user of the client device 302 before the user can operate the client device 302 for servicing the medical device 106. In some further examples, the method 900 can be performed to identify, authenticate, and authorize a user for servicing the medical device 106 when the medical device 106 is not connected to a network.

The method 900 includes an operation 902 of prompting a user (such as one of the service engineers SE, service personnel SP, or testing engineers TE) to enter a password for initiating a service software application 406 on the medical device 106. The user can access the service software application 406 through advanced settings on the medical device 106. When the user opens the service software application 406, the service software application 406 prompts the user to enter the password. In examples where the display screen 218 is a touchscreen, the prompt is displayed on the display screen 218 and the user can enter the password using the display screen 218. In alternative examples, the user can enter the password using one or more of the input devices on the client device 302. In some examples, the service software applications 406 included in the service software 402 share the same password. In other examples, the service software applications 406 can each have a unique password.

Next, the method 900 includes an operation 904 of determining whether the password received in operation 902 is correct. When the password is incorrect (i.e., “No” in operation 904), the method 900 can return to operation 902 and request that the user re-enter the password. When the password is correct (i.e., “Yes” in operation 904), the method 900 proceeds to an operation 906 of activating the authentication device 330.

In some examples, operation 906 includes activating a port on the medical device (e.g., the first port 308) in which the authentication device 330 is plugged into to activate the authentication device 330. In other examples, operation 906 includes detecting a wireless signal from the authentication device 330 such as a near-field communication (NFC) signal to detect the presence of the authentication device 330, and thereafter activating the authentication device 330. In further examples, operation 906 can require a user to touch a portion 334 on the authentication device 330 (see FIG. 3) for determining that the authentication device 330 is in the possession of a human operator, and thereafter activating the authentication device 330.

In further examples, the medical device 106 can detect when the authentication device 330 is removed from a port of the medical device 106, or is no longer proximal to the medical device 106 such that the medical device 106 no longer receives the wireless signal from the authentication device 330. When the medical device 106 detects that the authentication device 330 is removed or no longer proximal, the method 900 can immediately deny access to the service software applications 406 on the client device 302 (see operation 918) which can occur at any time during the method 900, or after completion of the method 900 when the user of the client device is granted access to the service software applications 406.

Next, the method 900 includes an operation 908 of prompting the user to enter the password 704 associated with the authentication device 330. In examples where the display screen 218 is a touchscreen, the prompt is displayed on the display screen 218 and the user can enter the password 704 using the display screen 218. In alternative examples, the user can enter the password 704 using one or more of the input devices on the client device 302. As described above, each authentication device 330 can have a unique password.

Next, the method 900 includes an operation 910 of determining whether the password received in operation 908 matches the password 704 stored on the authentication device 330. When the password does not match the password 704 stored on the authentication device 330 (i.e., “No” in operation 910), the method 900 can return to operation 908 and request that the user re-enter the password for the authentication device 330. When the password matches (i.e., “Yes” in operation 910), the method 900 proceeds to an operation 912 of acquiring the signed certificate 702 stored on the authentication device 330.

Next, the method 900 includes an operation 914 of verifying whether the signed certificate 702 stored on the authentication device 330 is valid. For example, operation 914 can include using the public key stored on the medical device 106 to validate the signed certificate 702. When the signed certificate 702 is determined to be valid (i.e., “Yes” in operation 914), the method 900 proceeds to an operation 916 of determining an accessibility level of the user. Otherwise, when the signed certificate 702 is determined to be invalid whether due to revocation, expiration, and the like (i.e., “No” in operation 914), the method 900 proceeds to an operation 918 of denying the user access to the service software application 406 on the medical device 106.

Operation 916 can include using the signed certificate 702 stored on the authentication device 330 to determine an accessibility level of the user. As described above, different types of signed certificates can be issued to different personnel based on a hierarchy, such as in the illustrative hierarchy shown in FIG. 8. Thus, the signed certificate 702 can be used by the medical device 106 to determine the user's accessibility level based credentials such as their position and/or role, level of training, and the like. In further examples, the accessibility level can also be restricted based on the geographical location of the clinical care environment 10 or the location of the medical device 106 within the clinical care environment.

In further examples, the signed certificate 702 can define a date range during which the signed certificate 702 is valid, and after which, the signed certificate 702 is expired. This can be done to verify that the signed certificate 702 remains authorized by the CA 802. In such examples, the medical device 106 can maintain a local real-time clock (RTC) which can be used to verify the validity of the signed certificate 702. In certain examples, the medical device 106 can be configured to manage the time such that the RTC cannot be rolled back by a malicious user to protect the integrity of the expiration date set for the signed certificate 702. Also, removing a user's authorization to access the service software application 406 can be accomplished by maintaining a certificate revocation list, which can include keeping track of the expiration dates for the signed certificate 702.

Next, the method 900 includes an operation 920 of determining whether the accessibility level of the user is appropriate for the service software application 406. When the accessibility level of the user indicates that the user has appropriate credentials and/or qualifications (i.e., “Yes” in operation 920), the method 900 proceeds to an operation 922 of granting access to the service software application 406. Otherwise, when the accessibility level of the user indicates that the user does not have appropriate credentials or qualifications for accessing the service software application 406 (i.e., “No” in operation 920), the method 900 proceeds to operation 918 of denying access to the service software application 406.

As an illustrative example, a service software application 406 can include a software testing and debugging application that is only accessible by the testing engineers TE. In such example, the testing engineers TE are provided with authentication devices 330 that include signed certificates 702 issued from the certificate authority (CA) 802 that permit the testing engineers TE to have access to the software testing and debugging application. The service engineers SE, service personnel SP, and biomedical equipment technicians, and other individuals who are provided with authentication devices 330, possess signed certificates 702 that do not permit these persons to have access to the software testing and debugging application. Additional examples for restricting access to the service software applications 406 based on the signed certificates 702 issued from the CA 802 are contemplated.

In view of the foregoing, the method 900 provides multiple layers of security: (1) requires a user to know the password to the service software application 406; (2) requires the user to have possession of the authentication device 330; (3) requires the user to know the password to the authentication device 330; (4) requires the authentication device 330 to have a valid signed certificate; and (5) requires the valid signed certificate on the authentication device 330 to have an appropriate accessibility level appropriate for the service software application 406. Additional layers of security may also be provided.

Further advantages of the method 900 include that the medical device 106 can be serviced off-line such that a connection to an authentication server is not required for authenticating a user for servicing the medical device 106. Instead, the medical device 106 is loaded with a public certificate for engineering access. In some examples, the medical device 106 is loaded with an additional healthcare delivery organization (HDO) certificate for local service. The authentication device 330 stores a signed certificate that identifies the user who has possession of the authentication device 330. The user can use the authentication device 330 an unlimited number of times until expiration or replacement of the signed certificate. This allows the medical device 106 to verify the user off-line such that the medical device 106 does not require a connection to an authentication server. Also, the signed certificate stored on the authentication device 330 is too large to manually enter. For example, the signed certificate can have a 2048-bit minimum key size. This can further increase the security for authenticating the user because the large size of the signed certificate makes it more difficult to forge.

FIG. 10 schematically illustrates an example of the medical device 106 that can be used to implement aspects of the present disclosure. As shown in the example provided in FIG. the medical device 106 includes one or more processing devices 1002, a memory storage device 1004, and a system bus 1006 that couples the memory storage device 1004 to the one or more processing devices 1002. The one or more processing devices 1002 can include central processing units (CPU). The processing device 1002 have a processing power and speed allowing the medical device 106 to perform servicing functions by itself, instead of requiring a separate, external device, such as the client device 302, to perform the servicing functions, such as the service software applications 406, which can require large amounts of computing capacity.

As shown in FIG. 10, the memory storage device 1004 can include a random-access memory (“RAM”) 1008 and a read-only memory (“ROM”) 1010. Basic input and output logic having basic routines that help to transfer information between elements within the medical device 106, such as during startup, can be stored in the ROM 1010.

The medical device 106 can also include a mass storage device 1012 that can include an operating system 1014 and store software instructions 1016 and data. The mass storage device 1012 is connected to the processing device 1002 through the system bus 1006. The mass storage device 1012 and associated computer-readable data storage media provide non-volatile, non-transitory storage for the medical device 106.

Although the description of computer-readable data storage media contained herein refers to the mass storage device 1012, it should be appreciated by those skilled in the art that computer-readable data storage media can be any available non-transitory, physical device or article of manufacture from which the medical device 106 can read data and/or instructions. The computer-readable storage media can be comprised of entirely non-transitory media. The mass storage device 1012 is an example of a computer-readable storage device.

Computer-readable data storage media include volatile and non-volatile, removable, and non-removable media implemented in any method or technology for storage of information such as computer-readable software instructions, data structures, program modules or other data. Example types of computer-readable data storage media include, but are not limited to, RAM, ROM, EPROM, EEPROM, flash memory or other solid-state memory technology, or any other medium which can be used to store information, and which can be accessed by the device.

The medical device 106 operates in a networked environment using logical connections to the other medical devices 106 and other electronic devices through the network 108. The medical device 106 connects to the network 108 through a network interface unit 1018 connected to the system bus 1006. The network interface unit 1018 can also connect to additional types of communications networks and devices, including through Bluetooth, Wi-Fi, and cellular telecommunications networks including 4G and 5G networks. The network interface unit 1018 may also connect the medical device 106 to additional networks, systems, and devices such as the EMR system 102, a digital health gateway, and other clinical resource centers.

The medical device 106 includes an input/output unit 1020 for receiving and processing inputs and outputs from a number of peripheral devices. Examples of peripheral devices can include, without limitation, a temperature probe, blood pressure cuffs, ECG or EKG leads, a clip for measuring blood oxygen saturation and pulse, and other peripheral devices.

The mass storage device 1012 and the RAM 1008 can store software instructions and data. The software instructions can include an operating system 1014 suitable for controlling the operation of the medical device 106. The mass storage device 1012 and/or the RAM 1008 can also store software instructions 1016, which when executed by the processing device 1002, cause the device to provide the functionality of the medical device 106 discussed herein.

The various embodiments described above are provided by way of illustration only and should not be construed to be limiting in any way. Various modifications can be made to the embodiments described above without departing from the true spirit and scope of the disclosure.

What is claimed is:

1. A medical device for assessing a health status of a patient, the medical device comprising:
one or more sensor modules each configured to obtain one or more physiological parameter measurements from the patient;
at least one processing device; and
a memory device storing:
firmware including:
service software for servicing the one or more sensor modules, the service software including an embedded web server; and
clinical software for acquiring the one or more physiological parameter measurements from the patient; and
instructions which, when executed by the at least one processing device, cause the at least one processing device to:
establish a server-client communications connection with a web browser on a client device; and
provide access on the web browser to the service software through the server-client communications connection, the access to the service software being provided without loading the service software onto the client device.

2. The medical device of claim 1, wherein the server-client communications connection enables the client device to control the one or more sensor modules.

3. The medical device of claim 1, wherein the server-client communications connection is established via Hypertext Transfer Protocol Secure (HTTPS).

4. The medical device of claim 1, wherein the service software includes one or more service software applications for calibration, functional verification tests, accuracy checks, component reporting, and log file display and download.

5. The medical device of claim 1, wherein access to the clinical software is blocked on the web browser.

6. The medical device of claim 5, wherein the access to the clinical software is provided on a built-in display of the medical device.

7. The medical device of claim 5, wherein the clinical software includes protected health information from the patient.

8. The medical device of claim 1, wherein the instructions further cause the at least one processing device to:
provide communication over an internet protocol between the client device and the medical device for live calibration of the one or more sensor modules.

9. The medical device of claim 1, wherein the server-client communications connection includes an Ethernet connection between the medical device and the client device.

10. The medical device of claim 1, wherein the server-client communications connection is a wireless connection between the medical device and the client device.

11. The medical device of claim 1, wherein the instructions further cause the at least one processing device to:
receive a password assigned to an authentication device;
receive a signed certificate stored on the authentication device;
validate the signed certificate by using a public key stored on the memory device; and
determine an accessibility level based on the signed certificate, wherein the accessibility level determines a level of access on the web browser to the service software.

12. The medical device of claim 11, further comprising:
at least one port for plugging the authentication device, and wherein the medical device receives the signed certificate via the at least one port.

13. The medical device of claim 11, wherein the medical device receives the signed certificate from the authentication device via a wireless signal.

14. The medical device of claim 11, wherein the instructions further cause the at least one processing device to:
block the access on the web browser to the service software when the authentication device is removed from a port of the medical device or is moved away such that the authentication device is no longer in close proximity to the medical device.

15. A medical device for assessing a health status of a patient, the medical device comprising:
at least one processing device; and
a memory device storing instructions which, when executed by the at least one processing device, cause the at least one processing device to:
receive a password assigned to an authentication device;
receive a signed certificate stored on the authentication device, the signed certificate created by a certificate authority;
validate the signed certificate by using a public key stored on the memory device;
determine an accessibility level based on the signed certificate;
establish a server-client communications connection between an embedded web server installed on the medical device and a web browser installed on a client device; and
provide access on the web browser to service software installed on the medical device through the server-client communications connection, the accessibility level determines a level of access on the web browser to the service software.

16. The medical device of claim 15, further comprising:
at least one port for plugging the authentication device, and wherein the medical device receives the signed certificate from a connection inside the at least one port.

17. The medical device of claim 15, wherein the medical device receives the signed certificate from the authentication device via a wireless signal.

18. The medical device of claim 15, wherein the instructions further cause the at least one processing device to:
block the access on the web browser to the service software when the authentication device is removed from a port of the medical device or when the authentication device is no longer in close proximity to the medical device.

19. The medical device of claim 15, wherein the server-client communications connection is established via Hypertext Transfer Protocol Secure (HTTPS).

20. A method of authenticating a user for servicing a medical device, the method comprising:
activating an authentication device;
receiving a signed certificate stored on the authentication device;
validating the signed certificate by using a public key; and
when the signed certificate is validated, determining an accessibility level based on the signed certificate for the user;
establishing a server-client communications connection between an embedded web server installed on the medical device and a web browser installed on a client device; and
providing access on the web browser to service software installed on the medical device through the server-client communications connection, wherein the accessibility level determines a level of access on the web browser to the service software.
